(19) United States
(12) Patent Application Publication
(10) Pub. No.: US 2018/0313290 A1
(43) Pub. Date: Nov. 1, 2018

(54) METHOD FOR ASCERTAINING A GAS CONCENTRATION IN A MEASURING GAS WITH THE AID OF A GAS SENSOR

(71) Applicant: Robert Bosch GmbH, Stuttgart (DE)

(72) Inventors: Thomas ZEIN, Sindelfingen (DE); Albrecht SCHMIDT, Kornwestheim (DE); Martin ELMER, Adlkofen (DE)

(21) Appl. No.: 15/770,614

(22) PCT Filed: Oct. 18, 2016
(86) PCT No.: PCT/EP2016/074971

§ 371 Date: Apr. 24, 2018

(30) Foreign Application Priority Data
Oct. 27, 2015 (DE) 10 2015 220 991.1

(51) Int. Cl. F02D 41/26; G01N 33/00; G01N 27/419

(52) U.S. Cl. CPC F02D 41/263; G01N 27/419; G01N 33/0036

(57) ABSTRACT

A method for ascertaining a gas concentration in a measuring gas with the aid of a gas sensor. In a first operating mode of an internal combustion engine, in which the gas concentration in the measuring gas is known, a plurality of value pairs of a respective gas concentration signal and a pressure signal are detected. Proceeding from these value pairs, a compensation parameter and a scaling factor of the gas sensor are ascertained. Subsequently, in a second operating mode of the internal combustion engine, the ascertainment of a gas concentration to be determined takes place, based on a gas concentration signal measured in the second operating mode of the internal combustion engine, and taking the compensation parameter and the scaling factor of the gas sensor into consideration.

### BACKGROUND INFORMATION

Conventional gas sensors and conventional operating methods for ascertaining gas concentrations in a measuring gas with the aid of gas sensors carry out a compensation of the dependence of the signals provided by the gas sensors on the absolute pressure in the measuring gas.

A method for ascertaining a gas concentration in a measuring gas with the aid of a gas sensor is described in German Patent Application No. DE 10 2006 011 837 A1, in which a gas concentration signal and a pressure signal are detected when a first operating mode of an internal combustion engine is present, in which the gas concentration in the measuring gas is known. It is furthermore provided there to ascertain a compensation parameter of the gas sensor proceeding from these signals. It is furthermore provided to then take the thus ascertained compensation parameter into consideration in at least one second operating mode of the internal combustion engine for the ascertainment of the gas concentration.

### SUMMARY

According to the present invention, a method for ascertaining a gas concentration in a measuring gas with the aid of a gas sensor is provided, in which in a first operating mode of an internal combustion engine, in which the gas concentration in the measuring gas is known, a plurality of value pairs of a respective gas concentration signal and a pressure signal are detected and, proceeding from these value pairs, a compensation parameter and a scaling factor of the gas sensor are ascertained, and in which subsequently, in a second operating mode of the internal combustion engine, the ascertainment of a gas concentration to be determined takes place, based on a gas concentration signal measured in the second operating mode of the internal combustion engine, and taking the previously ascertained compensation parameter and the scaling factor of the gas sensor into consideration.

A plurality within the scope of the present application is a natural number which is no smaller than 3, in particular even no smaller than 5. It is preferred that the method is carried out using a high number of value pairs, so that a plurality within the scope of the present application may in particular also be a natural number which is no smaller than 10.

The approach according to the present invention is more advantageous than the conventional approach since, based on the value pairs detected in the first operating mode, in addition to the compensation parameter a scaling factor of the gas sensor is also simultaneously ascertained, and thus an actual gas concentration may be ascertained more precisely in the second operating mode based on a subsequently measured gas concentration signal.

The compensation parameter and the scaling factor are in particular variables which are used to infer the actual gas concentration from the signals of a gas sensor (also: gas concentration signals), for example from a current supplied by the gas sensor or a voltage supplied by the gas sensor or a variable proportional thereto, for example a thus calculated assumed gas concentration. These variables may be slightly different for each individual gas sensor in a measuring gas having a given gas concentration, according to manufacturing variations and/or aging processes to which the individual gas sensor is subjected.

In a preferred refinement, the gas concentration signals measured in the first operating mode and in the second operating mode may be ascertained by gas sensors which are exposed to the measuring gas in the intake system of the internal combustion engine downstream from an exhaust gas recirculation valve. An exhaust gas recirculation valve of the internal combustion engine is preferably closed in the first operating mode, so that the gas sensor in this operating mode is exposed to a measuring gas in which the fraction of the gas concentration to be ascertained is as high as in the ambient air, i.e., in general 20.95%.

The compensation parameter and the scaling factor are preferably determined in an optimization process, for example by a fit process. For this purpose, the optimization process in particular minimizes the sum across all value pairs of the squares of the differences from the respective gas concentration signal and a predefined function which is dependent on the gas concentration signal and the pressure signal, and whose parameters are the compensation parameter and the scaling factor.

In particular, the determination takes place according to the following condition:

$${Min}_{\{ k , {m}_{adap} \}} [ {\sum }_{i = 1}^{N} {( {I}_{i} - I ( {p}_{i } ) )}^{2} ] , where I ( {p}_{i} ) = \frac{{I}_{i}}{{m}_{adap}} \frac{{p}_{i}}{k + {p}_{i}} \cdot \frac{k + {p}_{0}}{{p}_{0}}$$

p₀ being a reference pressure, k the compensation parameter, and m_{adap} the scaling factor, Iᵢ being a gas concentration signal and pᵢ a pressure signal, and N being the number of the detected value pairs.

Instead of the predefined function and the gas concentration signal, it is also possible to use the reciprocal values of these variables.

The reference pressure may be the normal pressure of 1013 mbar. However, another pressure may also be used as the reference pressure, which ultimately remains without significant effect on the overall process as long as the determination of the gas concentration in the second operating mode takes place based on the same reference pressure.

The above-described minimization problem may be solved in that both the derivative of the sum across all value pairs of the squares of the differences from the respective gas concentration signal and the predefined function with respect to the compensation parameter and the derivative of this sum with respect to the scaling factor are set to zero. As an alternative, it is also possible to form the sum across the squares of the differences of the reciprocal values of the respective gas concentration signal and the predefined function, and to set the derivative of this sum with respect to the compensation parameter to zero, and at the same time to set the derivative of this sum with respect to the scaling factor to zero.

Identical transformation then in particular results in:

$$( - {A}_{1} \cdot {B}_{2} + {A}_{2} \cdot {B}_{1} ) \cdot {k}^{3} + ( - {A}_{1} \cdot {B}_{2} \cdot {p}_{0} + {A}_{2} \cdot {B}_{3} - {A}_{3} \cdot {B}_{2} + {A}_{4} \cdot {B}_{1} ) \cdot {k}^{2} + ( - {A}_{3} \cdot {B}_{2} \cdot {p}_{0} + {A}_{4} \cdot {B}_{3} ) - {A}_{5} \cdot {B}_{2} + {A}_{6} \cdot {B}_{1} ) \cdot k + ( - {A}_{5} \cdot {B}_{2} \cdot {p}_{0} + {A}_{6} \cdot {B}_{3} ) = 0$$

$$and$$

$${m}_{adap} = \frac{{B}_{2} ( k + {p}_{0} )}{{B}_{1} \cdot k + {B}_{3}}$$

p₀ being the reference pressure, k the compensation parameter, and m_{adap} the scaling factor, and the coefficients in particular being calculated according to:

$${A}_{1} = {\sum }_{i} \frac{{p}_{0}^{2}}{\text{?}} ; {A}_{2} = - {\sum }_{i} \frac{\text{?}}{\text{?}} ; {A}_{3} = 2 {\sum }_{i} \frac{\text{?}}{{p}_{i}} ;$$

$${A}_{4} = - {\sum }_{i} \frac{{p}_{0}}{{p}_{i}} ( \text{?} + \text{?} ) \frac{1}{{I}_{1}} ; {A}_{5} = {\sum }_{i} \text{?} ; {A}_{6} = - {\sum }_{i} \frac{{p}_{0}^{2}}{{I}_{1}}$$

$${B}_{1} = {\sum }_{i} \frac{{p}_{0}^{2}}{\text{?}} ( {p}_{0} - {p}_{i} ) ; {B}_{2} = - {\sum }_{i} \frac{{p}_{0}}{{p}_{i}} ( {p}_{0} - {p}_{i} ) \frac{1}{{I}_{i}} ;$$

$${B}_{3} = {\sum }_{i} \frac{{p}_{0}^{2}}{{p}_{i}} ( \text{?} - {p}_{i} ) ; {B}_{4} = {p}_{0} \cdot {B}_{2}$$

$$\text{?} \text{indicates text missing or illegible when filed}$$

From the cubic equation, the compensation parameter may be rapidly solved numerically, for example with the aid of Newton's method. Using the thus ascertained compensation parameter, the scaling factor may then be determined.

The method may be carried out with the aid of an electronic control unit, which includes an electronic storage medium. It is very advantageously possible, in the first operating mode of the internal combustion engine, while the value pairs are being successively detected, to store only coefficients, for example a maximum of 10 different coefficients, in particular the above-defined coefficients, in the electronic storage medium, and to update these successively, in particular by summation. In particular, when a very high number of value pairs is to be taken into consideration for the determination of the compensation parameter and of the scaling factor, for example more than 30 value pairs, this results in a drastic reduction in the data to be stored in the electronic storage medium during the method.

Generally, a very large number of value pairs may be taken into consideration. Since the value pairs are continuously detected without further restrictions, even 1000 and more value pairs may be considered within a short time. Since the coefficients are successively adapted by the summands being added based on the new value pairs, the method may be carried out in such a way that the influence of earlier value pairs is basically continuously disregarded at a predefinable time constant.

Advantageously, the detected value pairs take pressure signals in the entire functionally relevant range into consideration, which extends from 500 mbar to 2000 mbar, or even to 2500 mbar, for example.

In the second operating mode of the internal combustion engine, for the ascertainment of the gas concentration in particular the variable I (pₘₑₐₛ) may be calculated, which is dependent on the gas concentration signal measured in the second operating mode and the pressure signal ascertained in the second operating mode, and whose parameters are the compensation parameter and the scaling factor, in particular according to the predefined function already mentioned above with respect to the first operating mode, in particular according to the formula

$$I ( {p}_{meas} ) = \frac{{I}_{nom}}{{m}_{adap}} \frac{{p}_{meas}}{k + {p}_{meas}} \cdot \frac{k + {p}_{0}}{{p}_{0}}$$

Iₙₒₘ being the gas concentration signal ascertained in the second operating mode, and p₀ being the reference pressure already mentioned above, k the compensation parameter, and m_{adap} the scaling factor, and Pₘₑₐₛ being the pressure signal ascertained in the second operating mode.

The pressure signal ascertained in the second operating mode may be the output signal of the gas sensor or of a further sensor, which is able to detect the pressure at the location or in the vicinity of the gas sensor.

The pressure signal ascertained in the second operating mode, however, may also be a variable which is ascertained, for example, by the electronic control unit with the aid of an exhaust gas air model.

### BRIEF DESCRIPTION OF THE DRAWINGS

Exemplary embodiments of the present invention are shown in the figures and are described in greater detail below.

FIG. 1 schematically shows the configuration of a gas sensor.

FIG. 2 shows value pairs ascertained according to the present invention.

FIG. 3 shows further value pairs ascertained according to the present invention.

### DETAILED DESCRIPTION OF EXAMPLE EMBODIMENTS

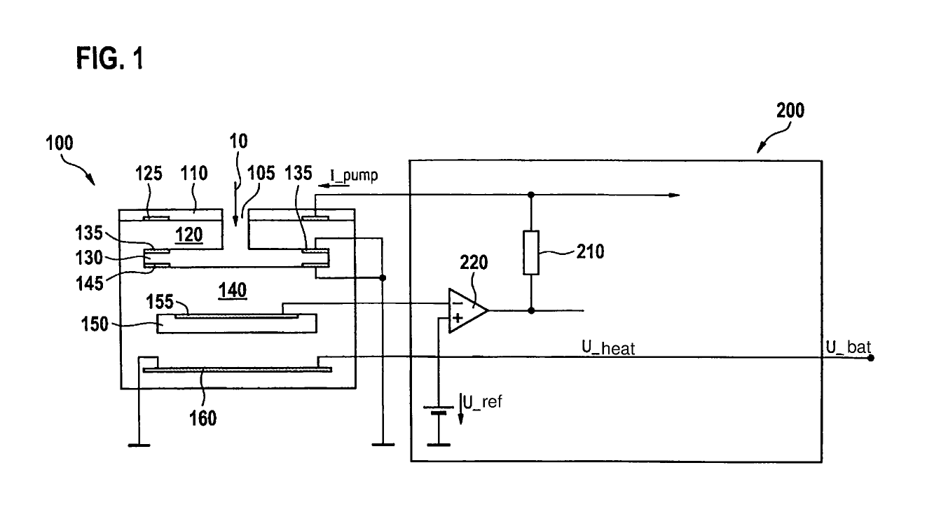

FIG. 1, by way of example, shows a gas sensor 100 for determining the concentration of gas components in a gas mixture, together with an associated device for activation 200. The gas sensor is designed as a broadband lambda sensor in the present example. It includes a heater 160 in a bottom area, a Nernst cell 140 in a central area, and a pump cell 120 in a top area. Pump cell 120 in a central area has an opening 105 through which exhaust gas 10 reaches a measuring chamber 130 of pump cell 120. Electrodes 135, 145 are situated at the outer ends of measuring chamber 130, upper electrodes 135 being assigned to the pump cell and forming interior pump electrodes (IPE) 135, and lower electrodes 145 being assigned to Nernst cell 140 and forming Nernst electrodes (NE) 145. The side of pump cell 120 facing the exhaust gas includes a protective layer 110 within which an exterior pump electrode (EPE) 125 is situated. A solid electrolyte, via which oxygen may be transported into measuring chamber 130 or transported out of measuring chamber 130 when a pump voltage is present at electrodes 125, 135, extends between exterior pump electrode 125 and interior pump electrode 135 of measuring chamber 130.

A further solid, which forms Nernst cell 140 including a reference gas chamber 150, adjoins pump cell 120. Reference gas chamber 150 is provided with a reference electrode (RE) 155 in the direction of the pump cell. The voltage taking effect between reference electrode 155 and Nernst electrode 145 in measuring chamber 130 of pump cell 120 corresponds to the Nernst voltage. During the further course of the ceramic, heater 160 is situated in a bottom area.

An oxygen reference gas is kept available in reference gas chamber 150 of Nernst cell 140. An oxygen concentration, which corresponds to a “lambda=1” concentration in measuring chamber 130, is set in the measuring chamber via a pump current flowing across pump electrodes 125 and 135.

An activation unit or control unit 200 assumes the control of these currents and the evaluation of the Nernst voltage. An operational amplifier 220 measures a Nernst voltage present at reference electrode 155 and compares this voltage to a reference voltage U_Ref, which typically is approximately 450 mV. In the event of deviations, operational amplifier 220 applies a pump current to pump cell 120 via a resistor 210 and pump electrodes 125, 135.

The method according to the present invention may, of course, also be carried out with the aid of other gas sensors 100; it is only essential in this respect that gas sensors 100 supply gas concentration signals which have a dependence on the absolute pressure of the measuring gas, as is also the case with broadband lambda sensors, for example, which include only a single electrochemical cell which may be operated as a pump cell, and as is also the case with NOx sensors generally including three electrochemical cells.

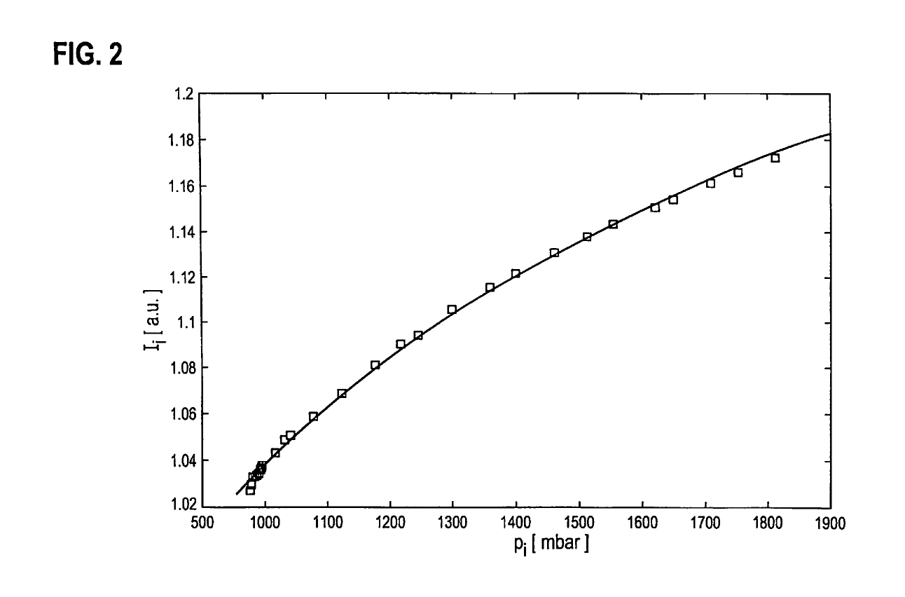

With the aid of gas sensor 100 according to FIG. 1, 28 value pairs Iᵢ, pᵢ including a respective value of absolute pressure pᵢ and associated gas concentration signal Iᵢ were ascertained in an intake system of an internal combustion engine downstream from a closed exhaust gas recirculation value, i.e., in ambient air whose oxygen content is 20.95%, in an interval of absolute pressures in the range from 950 mbar to 1900 mbar. These value pairs are represented in FIG. 2. Alternatively, the value pairs could also have been ascertained in a range extending from 500 mbar to 2500 mbar.

Based on these value pairs Iᵢ, pᵢ, initially coefficients A₁-A₆, B₁-B₄ were successively formed according to sum formulas

$${A}_{1} = {\sum }_{i} \frac{{p}_{0}^{2}}{\text{?}} ; {A}_{2} = - {\sum }_{i} \frac{\text{?}}{\text{?}} ; {A}_{3} = 2 {\sum }_{i} \frac{\text{?}}{{p}_{i}} ;$$

$${A}_{4} = - {\sum }_{i} \frac{{p}_{0}}{{p}_{i}} ( \text{?} + \text{?} ) \frac{1}{{I}_{1}} ; {A}_{5} = {\sum }_{i} \text{?} ; {A}_{6} = - {\sum }_{i} \frac{{p}_{0}^{2}}{{I}_{1}}$$

$${B}_{1} = {\sum }_{i} \frac{{p}_{0}^{2}}{\text{?}} ( {p}_{0} - {p}_{i} ) ; {B}_{2} = - {\sum }_{i} \frac{{p}_{0}}{{p}_{i}} ( {p}_{0} - {p}_{i} ) \frac{1}{{I}_{i}} ;$$

$${B}_{3} = {\sum }_{i} \frac{{p}_{0}^{2}}{{p}_{i}} ( \text{?} - {p}_{i} ) ; {B}_{4} = {p}_{0} \cdot {B}_{2}$$

$$\text{?} \text{indicates text missing or illegible when filed}$$

progressively with the detection of the value pairs, p₀ being a reference pressure, for example the normal pressure of 1013 mbar. For this purpose, coefficients A₁-A₆, B₁-B₄ were stored in an electronic storage medium of an electronic control unit and incrementally modified with each further detected value pair Iᵢ, pᵢ by adding up the respective terms. Permanent storage of value pairs Iᵢ, pᵢ, in contrast, did not take place, conserving resources.

Subsequently, compensation parameter k was determined from coefficients A₁-A₆, B₁-B₄. For this purpose, the cubic equation for compensation parameter k

(−A₁·B₂+A₂·B₁)·k³+(−A₁·B₂·p₀+A₂·B₃−A₃·B₂+A₄·B₁)·k²+(−A₃·B₂·p₀+A₄·B₃−A₅·B₂+A₆·B₁)·k+(−A₅·B₂p₀+A₆·B₃)=0

was solved with the aid of Newton's method. Thereafter, scaling factor m_{adap} was calculated according to the formula

$${m}_{adap} = - \frac{{B}_{1} ( k + \text{?} )}{{B}_{1} \cdot k + \text{?}} . \\ \text{?} \text{indicates text missing or illegible when filed}$$

In this connection, it shall be emphasized as very advantageous that the memory space occupancy for the ascertainment of compensation parameter k and of scaling factor m_{adap} is independent of the number of value pairs Iᵢ, pᵢ used for this purpose.

In the subsequent second operating mode of the internal combustion engine, gas concentration signals Iₙₒₘ were continuously ascertained with the aid of the same gas sensor. During this time, the exhaust gas recirculation valve was open, partially open and closed in a not fixedly predefined manner as required by the operation of the internal combustion engine.

The oxygen concentration in the measuring gas exposed to the lambda sensor, i.e., the air supplied to the internal combustion engine, fluctuated accordingly. Furthermore, pressure signal pₘₑₐₛ ascertained in the second operating mode at the location of the gas sensor with the aid of a pressure sensor fluctuated.

Proceeding from gas concentration signal Iₙₒₘ measured in the second operating mode, with the aid of the formula

$$I ( {p}_{meas} ) = \frac{{I}_{nom}}{{m}_{adap}} \frac{{p}_{meas}}{k + {p}_{meas}} \cdot \frac{k + {p}_{0}}{{p}_{0}}$$

the actual oxygen concentration was always determined with high accuracy.

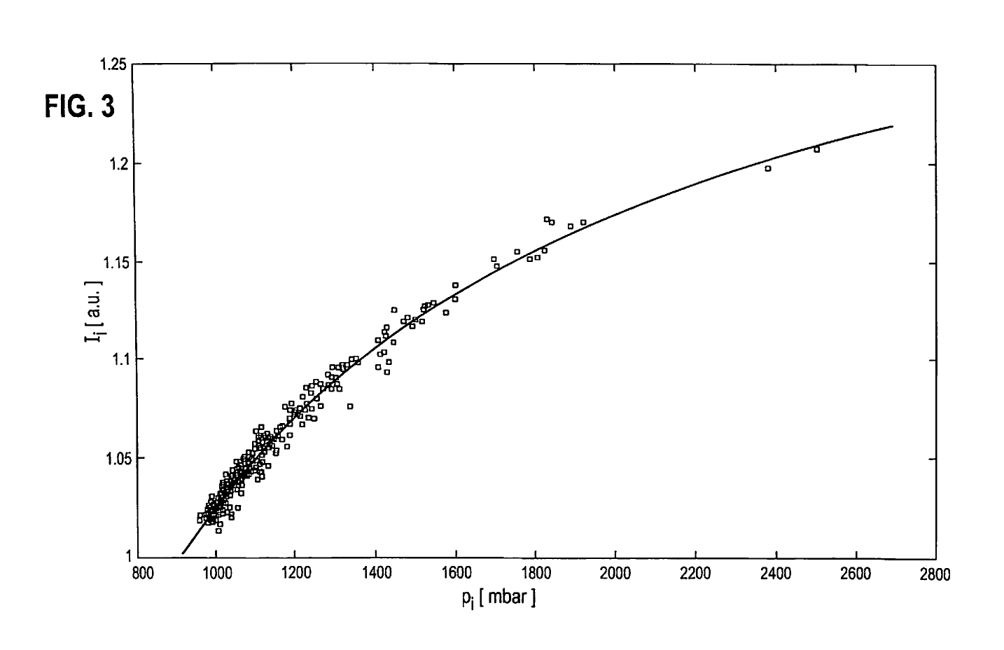

FIG. 3 shows a further example of value pairs Iᵢ, pᵢ ascertained according to the present invention, with which the method may also be carried out. The number of value pairs Iᵢ, pᵢ is much larger compared to FIG. 2. However, even with the many value pairs Iᵢ, pᵢ, the computing complexity required for the determination of the actual oxygen concentration increases at most only proportionally to the number of the value pairs. It may thus be comfortably achieved incrementally with the occurrence of value pairs Iᵢ, pᵢ. The memory space required in the control unit remains unchanged.

1-15. (canceled)

16. A method for ascertaining a gas concentration in a measuring gas using a gas sensor, the method comprising:
in a first operating mode of an internal combustion engine, in which the gas concentration in the measuring gas is known, detecting a plurality of value pairs of a respective gas concentration signal and a pressure signal;
ascertaining from the value pairs, a compensation parameter and a scaling factor of the gas sensor; and
in a second operating mode of the internal combustion engine, after the first operating mode, ascertaining a gas concentration based on a gas concentration signal measured in the second operating mode of the internal combustion engine, and taking the compensation parameter and the scaling factor of the gas sensor into consideration.

17. The method as recited in claim 16, wherein an exhaust gas recirculation valve of the internal combustion engine is closed in the first operating mode of the internal combustion engine.

18. The method as recited in claim 16, wherein in the first operating mode, the compensation parameter and the scaling factor are determined in an optimization process, so that the following variable assumes a minimum: a sum across all of the value pairs of squares of differences from the respective gas concentration signal or its reciprocal value and a predefined function or of its reciprocal value, the predefined function being dependent on the gas concentration signal and the pressure signal, and the compensation parameter and the scaling factor being parameters of the predefined function.

19. The method as recited in claim 16, wherein, in the first operating mode, the compensation parameter and the scaling factor are determined as solutions of two equations, the gas concentration signals and the pressure signals being incorporated in the equations only indirectly via coefficients, which are formed by summing terms dependent on the gas concentration signals and the pressure signals.

20. The method as recited in claim 18, wherein, in the second operating mode of the internal combustion engine, for the ascertainment of the gas concentration the value of a second predefined function is calculated, which is dependent on the gas concentration signal measured in the second operating mode and the pressure signal ascertained in the second operating mode, and whose parameters are the compensation parameter and the scaling factor.

21. The method as recited in claim 20, wherein the predefined function and the second predefined function are identical.

22. The method as recited in claim 16, wherein, in the first operating mode, the compensation parameter and the scaling factor are determined from the value pairs according to the condition:
$${Min}_{\{ k , {m}_{adap} \}} [ {\sum }_{i = 1}^{N} {( {I}_{i} - I ( {p}_{i } ) )}^{2} ] , where I ( {p}_{i} ) = \frac{{I}_{i}}{{m}_{adap}} \frac{{p}_{i}}{k + {p}_{i}} \cdot \frac{k + {p}_{0}}{{p}_{0}}$$
p₀ being a reference pressure, k being the compensation parameter, and m_{adap} being the scaling factor, and N being the number of the detected value pairs.

23. The method as recited in claim 16, wherein, in the first operating mode, the compensation parameter and the scaling factor are determined as a solution of a first equation:
(−A₁·B₂+A₂·B₁)·k³+(−A₁·B₂·p₀+A₂·B₃−A₃·B₂+A₄·B₁)·k²+(−A₃·B₂·p₀+A₄·B₃)−A₅·B₂+A₆·B₁)·k+(−A₅·B₂p₀+A₆·B₃)=0
and of a second equation:
$${m}_{adap} = - \frac{{B}_{1} ( k + \text{?} )}{{B}_{1} \cdot k + \text{?}}$$
$$\text{?} \text{indicates text missing or illegible when filed}$$
p₀ being a reference pressure, k being the compensation parameter, and m_{adap} being the scaling factor, and coefficients (A₁-A₆, B₁-B₄) being calculated according to:
$${A}_{1} = {\sum }_{i} \frac{{p}_{0}^{2}}{\text{?}} ; {A}_{2} = - {\sum }_{i} \frac{\text{?}}{\text{?}} ; {A}_{3} = 2 {\sum }_{i} \frac{\text{?}}{{p}_{i}} ;$$
$${A}_{4} = - {\sum }_{i} \frac{{p}_{0}}{{p}_{i}} ( \text{?} + \text{?} ) \frac{1}{{I}_{1}} ; {A}_{5} = {\sum }_{i} \text{?} ; {A}_{6} = - {\sum }_{i} \frac{{p}_{0}^{2}}{{I}_{1}}$$
$${B}_{1} = {\sum }_{i} \frac{{p}_{0}^{2}}{\text{?}} ( {p}_{0} - {p}_{i} ) ; {B}_{2} = - {\sum }_{i} \frac{{p}_{0}}{{p}_{i}} ( {p}_{0} - {p}_{i} ) \frac{1}{{I}_{i}} ;$$
$${B}_{3} = {\sum }_{i} \frac{{p}_{0}^{2}}{{p}_{i}} ( \text{?} - {p}_{i} ) ; {B}_{4} = {p}_{0} \cdot {B}_{2}$$
$$\text{?} \text{indicates text missing or illegible when filed}$$

24. The method as recited in claim 23, wherein the first equation is solved numerically according to Newton's method.

25. The method as recited in claim 22, wherein, in the second operating mode of the internal combustion engine, for the ascertainment of the gas concentration the variable I(pₘₑₐₛ) is calculated according to the formula
$$I ( {p}_{meas} ) = \frac{{I}_{nom}}{{m}_{adap}} \frac{{p}_{meas}}{k + {p}_{meas}} \cdot \frac{k + {p}_{0}}{{p}_{0}}$$
Iₙₒₘ being the gas concentration signal ascertained in the second operating mode, and p₀ being a reference pressure, k the compensation parameter, and m_{adap} the scaling factor.

26. The method as recited in claim 16, wherein, in the second operating mode of the internal combustion engine, a gas concentration in the intake system of the internal combustion engine is ascertained, downstream from an exhaust gas recirculation valve.

27. An non-transitory electronic storage medium on which is stored a computer program for ascertaining a gas concentration in a measuring gas using a gas sensor, the computer program, when executed by an electronic control unit, causing the electronic control unit to perform:
in a first operating mode of an internal combustion engine, in which the gas concentration in the measuring gas is known, detecting a plurality of value pairs of a respective gas concentration signal and a pressure signal;
ascertaining from the value pairs, a compensation parameter and a scaling factor of the gas sensor; and
in a second operating mode of the internal combustion engine, after the first operating mode, ascertaining a gas concentration based on a gas concentration signal measured in the second operating mode of the internal combustion engine, and taking the compensation parameter and the scaling factor of the gas sensor into consideration.

28. An electronic control unit, which includes a non-transitory electronic storage medium on which is stored a computer program for ascertaining a gas concentration in a measuring gas using a gas sensor, the computer program, when executed by an electronic control unit, causing the electronic control unit to perform:
in a first operating mode of an internal combustion engine, in which the gas concentration in the measuring gas is known, detecting a plurality of value pairs of a respective gas concentration signal and a pressure signal;
ascertaining from the value pairs, a compensation parameter and a scaling factor of the gas sensor; and
in a second operating mode of the internal combustion engine, after the first operating mode, ascertaining a gas concentration based on a gas concentration signal measured in the second operating mode of the internal combustion engine, and taking the compensation parameter and the scaling factor of the gas sensor into consideration.

29. An electronic control unit, including an non-transitory electronic storage medium on which is stored a computer program for ascertaining a gas concentration in a measuring gas using a gas sensor, the computer program, when executed by an electronic control unit, causing the electronic control unit to perform:
in a first operating mode of an internal combustion engine, in which the gas concentration in the measuring gas is known, detecting a plurality of value pairs of a respective gas concentration signal and a pressure signal;
ascertaining from the value pairs, a compensation parameter and a scaling factor of the gas sensor; and
in a second operating mode of the internal combustion engine, after the first operating mode, ascertaining a gas concentration based on a gas concentration signal measured in the second operating mode of the internal combustion engine, and taking the compensation parameter and the scaling factor of the gas sensor into consideration;
wherein, in the first operating mode, the compensation parameter and the scaling factor are determined as a solution of a first equation:
(−A₁·B₂+A₂·B₁)·k³+(−A₁·B₂·p₀+A₂·B₃−A₃·B₂+A₄·B₁)·k²+(−A₃·B₂·p₀+A₄·B₃)−A₅·B₂+A₆·B₁)·k+(−A₅·B₂p₀+A₆·B₃)=0
and of a second equation:
$${m}_{adap} = - \frac{{B}_{1} ( k + \text{?} )}{{B}_{1} \cdot k + \text{?}} . \\ \text{?} \text{indicates text missing or illegible when filed}$$
p₀ being a reference pressure, k being the compensation parameter, and m_{adap} being the scaling factor, and coefficients (A₁-A₆, B₁-B₄) being calculated according to:
$${A}_{1} = {\sum }_{i} \frac{{p}_{0}^{2}}{\text{?}} ; {A}_{2} = - {\sum }_{i} \frac{\text{?}}{\text{?}} ; {A}_{3} = 2 {\sum }_{i} \frac{\text{?}}{{p}_{i}} ;$$
$${A}_{4} = - {\sum }_{i} \frac{{p}_{0}}{{p}_{i}} ( \text{?} + \text{?} ) \frac{1}{{I}_{1}} ; {A}_{5} = {\sum }_{i} \text{?} ; {A}_{6} = - {\sum }_{i} \frac{{p}_{0}^{2}}{{I}_{1}}$$
$${B}_{1} = {\sum }_{i} \frac{{p}_{0}^{2}}{\text{?}} ( {p}_{0} - {p}_{i} ) ; {B}_{2} = - {\sum }_{i} \frac{{p}_{0}}{{p}_{i}} ( {p}_{0} - {p}_{i} ) \frac{1}{{I}_{i}} ;$$
$${B}_{3} = {\sum }_{i} \frac{{p}_{0}^{2}}{{p}_{i}} ( \text{?} - {p}_{i} ) ; {B}_{4} = {p}_{0} \cdot {B}_{2}$$
$$\text{?} \text{indicates text missing or illegible when filed}$$
wherein coefficients (A₁-A₆, B₁-B₄) are stored in the control unit, in particular in the electronic storage medium.
